(19) United States
(12) Patent Application Publication
(10) Pub. No.: US 2024/0277889 A1
(43) Pub. Date: Aug. 22, 2024

(54) DEEP TISSUE IN VIVO PRINTING

(71) Applicant: California Institute of Technology, Pasadena, CA (US)

(72) Inventors: Wei GAO, San Marino, CA (US); Elham DAVOODI, Los Angeles, CA (US)

(21) Appl. No.: 18/444,514

(22) Filed: Feb. 16, 2024

Related U.S. Application Data
(60) Provisional application No. 63/446,122, filed on Feb. 16, 2023.

(51) Int. Cl. A61L 24/00; A61K 9/127; A61M 37/00; B33Y 10/00; B33Y 30/00; B33Y 50/00; B33Y 70/00

(52) U.S. Cl. CPC A61L 24/0015; A61K 9/127; A61L 24/0094; A61M 37/0092; B33Y 10/00; B33Y 30/00; B33Y 50/00; B33Y 70/00; A61L 2400/06; A61M 2205/058

(57) ABSTRACT

Techniques for deep tissue in vivo printing are described. Some implementations of the disclosure relate to a method that includes: obtaining a biopolymer mixture including prepolymer material and a crosslinking agent encapsulated in carrier particles; delivering the biopolymer mixture to a subcutaneous or deep tissue target location of a subject; and transmitting with a bioprinting device, via transcutaneous application, radiation to the subcutaneous or deep tissue target location, the radiation configured to cause the carrier particles to release at least some of the crosslinking agent, the released crosslinking agent configured to cause the prepolymer material to form into a gel or polymeric matrix.

### CROSS-REFERENCE TO RELATED APPLICATIONS

This application claims the benefit of U.S. Provisional Patent Application No. 63/446,122, filed Feb. 16, 2023, and titled “Deep Tissue In vivo Printing”, which is incorporated herein by reference in its entirety.

### BACKGROUND

The majority of developed three-dimensional (3D) bioprinting techniques involve ex-vivo printing of biomaterials, followed by implanting the patterned biomaterial in a target site through open surgery. Some recent novel techniques have enabled in-situ printing of the biomaterial directly on the tissue. Although this eliminates issues related to handling delicate patterns and enables further bioprinting applications, the need for open surgery remains a major issue. Open surgeries can be associated with several complications, including complications from using anesthesia, long recovery periods, and death.

Noninvasive in vivo printing aims to eliminate the need for invasive operations by taking advantage of novel technologies for deposition and crosslinking of bioinks. Some current in vivo printing techniques involve subcutaneous injection of a light-sensitive biomaterial that is exposed to a near-infrared (NIR) laser to trigger gelation. This approach has several advantages over conventional bioprinting techniques, including that there is no need to access the site of printing. However, due to the limited penetration of NIR light, this approach is limited to printing to regions a few millimeters under the skin.

### SUMMARY

The technology described herein relates to deep tissue in vivo bioprinting.

In one embodiment, a method comprises: obtaining a biopolymer mixture including prepolymer material and a crosslinking agent encapsulated in carrier particles; delivering the biopolymer mixture to a subcutaneous or deep tissue target location of a subject; and transmitting with a bioprinting device, via transcutaneous application, radiation to the subcutaneous or deep tissue target location, the radiation configured to cause the carrier particles to release at least some of the crosslinking agent, the released crosslinking agent configured to cause the prepolymer material to form into a gel or polymeric matrix.

In some implementations, the biopolymer mixture further includes a contrast agent; and the method further comprises: capturing, using an imaging device, an image of the subcutaneous or deep tissue target location of the subject, the image enhanced by the contrast agent.

In some implementations, the carrier particles comprise vesicles, micelles, bubbles, or polymers. In some implementations, the prepolymer material comprises alginate, polyethylene glycol diacrylate (PEGDA), or polydimethylsiloxane (PDMS).

In some implementations, the vesicles comprise liposomes encapsulating the crosslinking agent; and the radiation is configured to cause the liposomes to increase in temperature and release the crosslinking agent.

In some implementations, the bioprinting device comprises one or more high-intensity focused ultrasound (HIFU) transducers; and transmitting with the bioprinting device, via transcutaneous application, the radiation to the subcutaneous or deep tissue target location comprises: transmitting with the one or more HIFU transducers, HIFU to the subcutaneous or deep tissue target location, the HIFU configured to cause the carrier particles to increase in temperature and release at least some of the crosslinking agent.

In some implementations, the HIFU is configured to heat the subcutaneous or deep tissue target location to between 39° C. and 43° C.

In some implementations, the prepolymer material is loaded with drugs; and the gel formed from the prepolymer material is configured, in the subcutaneous or deep tissue target location, to provide a controlled release of the drugs for a therapeutic application.

In some implementations, the prepolymer material is loaded with cells; and the gel formed from the prepolymer material is configured, in the subcutaneous or deep tissue target location, to encapsulate the cells for tissue regeneration.

In some implementations, the prepolymer material is electrically conductive; and the gel formed from the prepolymer material is electrically conductive.

In some implementations, the prepolymer material is bioadhesive; and the gel formed from the prepolymer material is configured to seal an internal wound of the subject at the subcutaneous or deep tissue target location.

In some implementations, transmitting with the bioprinting device, via transcutaneous application, the radiation to the subcutaneous or deep tissue target location comprises: transmitting with one or more transducers of the bioprinting device, along a predetermined trajectory, the radiation to the subcutaneous or deep tissue target location to cause the prepolymer material to form into a pattern of the gel defined by the predetermined trajectory.

In one embodiment, a system for in vivo bioprinting comprises: a biopolymer mixture including prepolymer material and a crosslinking agent encapsulated in carrier particles; and a bioprinting device configured to transmit, via transcutaneous application, radiation to a subcutaneous or deep tissue target location of a subject that the biopolymer mixture is delivered to, the radiation configured to cause the carrier particles to release at least some of the crosslinking agent, the released crosslinking agent configured to cause the prepolymer material to form into a gel or polymeric matrix.

In some implementations, the biopolymer mixture further includes a contrast agent; and the system further comprises an imaging device configured to capture an image of the subcutaneous or deep tissue target location of the subject after the biopolymer mixture is delivered to the subcutaneous or deep tissue target location.

In some implementations, the bioprinting device comprises one or more HIFU transducers, the one or more HIFU transducers configured to transmit the radiation as HIFU to the subcutaneous or deep tissue target location, the HIFU configured to cause the carrier particles to release at least some of the crosslinking agent; and the imaging device comprises an ultrasound imaging device.

In some implementations, the bioprinting device of the system comprises: one or more transducers configured to the transmit the radiation to the subcutaneous or deep tissue target location; and a controller configured to cause the one or transducers to transmit, along a predetermined trajectory, the radiation to the subcutaneous or deep tissue target location to cause the prepolymer material to form into a pattern of the gel defined by the predetermined trajectory.

In some implementations, the bioprinting device comprises an array of transducers including the one or more transducers; and the controller is configured to cause the transducers of the array of transducers to operate in an order associated with the predetermined trajectory.

In some implementations, the bioprinting device comprises a motor configured to position the one or more transducers in relation to the subcutaneous or deep tissue target location; and the controller is configured to control the motor to position the one or more transducers to transmit, along the predetermined trajectory, the radiation to the subcutaneous or deep tissue target location.

In some implementations, the carrier particles comprise temperature sensitive particles (e.g., vesicles) configured to release the crosslinking agent in response to a temperature increase; and the crosslinking agent is configured to induce thermal crosslinking of the prepolymer material.

In some implementations, the prepolymer material is loaded with drugs, and the gel formed from the prepolymer material is configured to provide a controlled release of the drugs in the subcutaneous or deep tissue target location; or the prepolymer material is loaded with cells, and the gel formed from the prepolymer material is configured to encapsulate the cells for tissue regeneration in the subcutaneous or deep tissue target location; or the prepolymer material is electrically conductive, and the gel formed from the prepolymer material is electrically conductive; or the prepolymer material is bioadhesive, and the gel formed from the prepolymer material is configured to seal an internal wound of the subject at the subcutaneous or deep tissue target location.

In one embodiment, a biopolymer mixture for in vivo bioprinting comprises: a contrast agent configured to enhance an image of the biopolymer mixture; a crosslinking agent encapsulated in carrier particles, the carrier particles configured to release the crosslinking agent in response to a temperature increase; and a prepolymer material configured to form into a gel when exposed to the crosslinking agent.

In some implementations, the carrier particles comprise vesicles, micelles, bubbles, or polymers. In some implementations, the prepolymer material comprises alginate, PEGDA, or PDMS. In some implementations, the carrier particles are temperature sensitive liposomes encapsulating the crosslinking agent.

Other features and aspects of the disclosed technology will become apparent from the following detailed description, taken in conjunction with the accompanying drawings, which illustrate, by way of example, the features in accordance with implementations of the disclosed technology. The summary is not intended to limit the scope of any inventions described herein, which are defined by the claims and equivalents.

### BRIEF DESCRIPTION OF THE DRAWINGS

The present disclosure, in accordance with one or more implementations, is described in detail with reference to the following figures. The figures are provided for purposes of illustration only and merely depict example implementations. Furthermore, it should be noted that for clarity and ease of illustration, the elements in the figures have not necessarily been drawn to scale.

FIG. 1 illustrates an example system for deep tissue in vivo printing in a subject, in accordance with some implementations of the disclosure.

FIG. 2 is a schematic diagram showing lipid bilayer changes of a liposome carrier upon heating the lipid bilayer from a solid state to a liquid state.

FIG. 3 illustrates example components that can be included with an in vivo bioprinting device to enable deep tissue in vivo printing, in accordance with some implementations of the disclosure.

FIG. 4 schematically illustrates the tissue penetration depth that can be achieved by different types of light radiation as contrasted with ultrasound.

FIG. 5 schematically illustrates how an ultrasound focal point of HIFU can be used to define a high printing resolution, in accordance with some implementations of the disclosure.

FIG. 6A is a schematic diagram illustrating an application of the in vivo printing technology described herein in wound sealing.

FIG. 6B is a schematic diagram illustrating an application of the in vivo printing technology described herein in bioelectronics.

FIG. 6C is a schematic diagram illustrating an application of the in vivo printing technology described herein in drug delivery and tissue regeneration.

FIG. 7 is an operational flow diagram illustrating example operations performed by a system for deep tissue in vivo printing in a subject, in accordance with some implementations of the disclosure.

FIG. 8 includes two plots showing calcium release over time upon heating liposomal solutions at 37° C. and 43° C.

FIG. 9 includes a plot showing the calcium release from liposomes after 6 months of storage at 25° C. and 4° C.

FIG. 10 includes a plot corresponding to a rheological study showing heat-trigged gelation of an alginate prepolymer for various liposome concentrations.

FIG. 11 shows images of various PDMS patterns 3D printed using HIFU, in accordance with some implementations of the disclosure.

FIG. 12 includes a plot showing power (Watt) and printing speed (mm/min) conditions leading to gel formation in the case of alginate prepolymer mixed with 50 wt. % CaCl2-encapsulated liposomes, in accordance with some implementations of the disclosure.

FIG. 13 includes a plot showing 3D bioprinting resolutions obtained in the case of alginate prepolymer mixed with 50 wt. % CaCl2-encapsulated liposomes using various power and printing speeds, in accordance with some implementations of the disclosure.

FIG. 14 illustrate PEGDA 3D printed by ultrasound exposure.

FIG. 15A includes a plot showing a resistance change for various line widths of a conductive hydrogel 3D printed using HIFU.

FIG. 15B includes a plot showing the ratio of resistance changes upon heating to different temperatures of a conductive hydrogel 3D printed using HIFU.

FIG. 15C includes plots showing stability under compression and tension of a conductive hydrogel 3D printed using HIFU.

FIG. 16A shows small molecule release percentage as a function of time from HIFU induced 3D printed drug-loaded hydrogels under acidic and neutral pH.

FIG. 16B shows large molecule release percentage as a function of time from HIFU induced 3D printed drug-loaded hydrogels under acidic and neutral pH.

FIG. 17A includes an image illustrating an injection site of a biopolymer mixture including a prepolymer under the mouse's skin.

FIG. 17B includes an image illustrating the results of ultrasound exposure and 3D printing of a pattern of the prepolymer injected in FIG. 17A.

FIG. 18A is a schematic diagram showing in vivo printing inside a bladder of a mouse model.

FIG. 18B includes an ultrasound image of a bladder of a mouse model undergoing in vivo printing to form a gel from a prepolymer.

FIG. 18C includes an image showing the bladder of FIG. 18B being filled with the prepolymer using a catheter.

FIG. 18D includes an image showing the gel and bladder of FIG. 18B being extracted.

The figures are not exhaustive and do not limit the present disclosure to the precise form disclosed.

### DETAILED DESCRIPTION

As used herein, the term “radiation” is intended to refer to the transmission or emission of energy in the form of particles or waves through space or a material medium. For example, radiation can refer to acoustic radiation such as ultrasound or audible sound. As another example, radiation can refer to electromagnetic radiation such as radio waves, infrared (IR), visible light, ultraviolet, x-rays, etc.

The technology described herein is directed to systems and methods for deep tissue in vivo bioprinting, including deep tissue gelation and patterning of biomaterials. To this end, the technology described herein leverages a biopolymer mixture that includes a prepolymer and crosslinking agents encapsulated in carrier particles (e.g., carrier vesicles). During use, the biopolymer mixture is injected or otherwise delivered to a target location (e.g., a deep tissue location). Thereafter, external radiation is applied to the target location, causing the carrier particles to release the crosslinking agents, and triggering on-demand gelation of the prepolymer. Particular implementations of the technology described herein are directed to using a HIFU radiation source that is capable of delivering deep tissue thermal radiation to thermosensitive carrier particles, triggering thermal release of the crosslinking agents.

Various advantages can be realized by the technology described herein. By virtue of encapsulating the crosslinking agents in carrier particles that can be triggered via external radiation, deep tissue bioprinting without the requirement of open surgery is enabled. In addition, a focused radiation source can be moved along a trajectory, enabling on-demand gelation and patterning of the prepolymer in regions of interest while leaving unexposed regions uncrosslinked. In addition, the technology described herein can be adapted to print a variety of biomaterials having various properties, including bioadhesion, conductivity, drug-delivery, etc. Various prepolymers in combination with crosslinking agents having different crosslinking mechanisms (e.g., thermal, ionic, free-radical polymerization, etc.) can be adapted for use with the technology described herein, enabling a large variety of possible uses.

These and other benefits realized by implementing the technology described herein are further describe below.

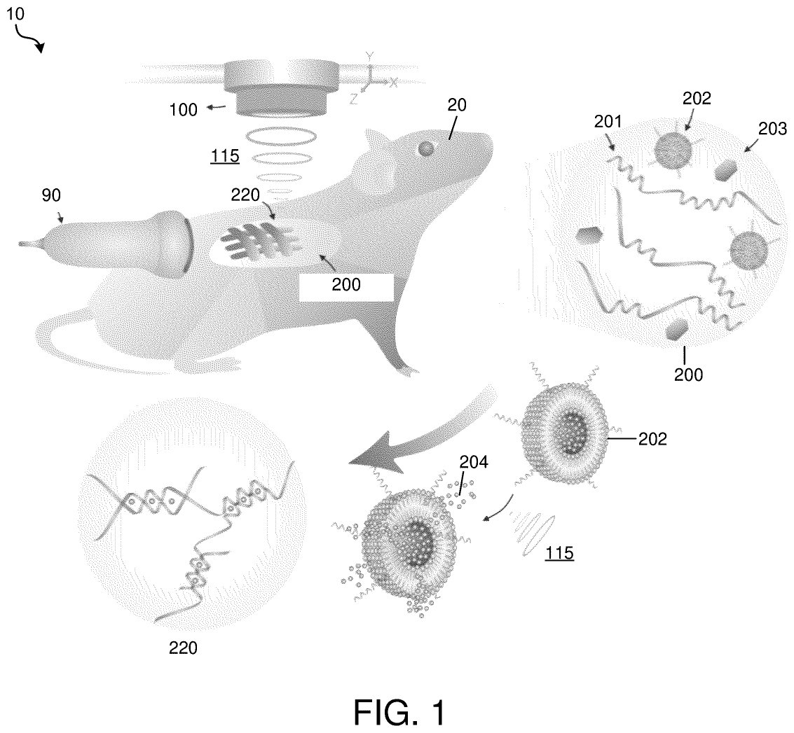

FIG. 1 illustrates an example system 10 for deep tissue in vivo printing in a subject 20, in accordance with some implementations of the disclosure. The system 10 includes an imaging device 90, an in vivo bioprinting device 100, and a biopolymer mixture 200. The biopolymer mixture 200 includes prepolymer material 201 and carrier particles 202. The carrier particles 202 encapsulate crosslinking agents 204 that are configured to cause prepolymer material 201 to gelate. The biopolymer mixture 200 can also include a contrast agent 203 that is used to enhanced images captured by imaging device 90.

During an in vivo bioprinting procedure, the biopolymer mixture 200 is delivered in a subcutaneous or deep tissue target location inside the body of subject 20. Thereafter, one or more transducers of in vivo bioprinting device 100 externally (i.e., by transcutaneous application) deliver radiation 115 to the target location containing the delivered biopolymer mixture 200. In response to the radiation, the carrier particles 202 are configured to release at least some of the crosslinking agents 204 to induce local crosslinking and gelation of prepolymer material 201, forming gel 220.

As depicted by FIG. 1, the bioprinting device 100 can scan over the biopolymer mixture 200 based on a predefined trajectory to induce local crosslinking that patterns the gel 220 in a desired manner. To this end, the bioprinting device 100 can utilize a control system. That control system can include a non-transitory computer readable medium having instructions stored thereon that are executable by a processor of the bioprinting device 100 to cause the one or more transducers of bioprinting device 100 to apply radiation in a predefined manner to effectuate bioprinting in a desired pattern. For example, the bioprinting device 100 can scan over the biopolymer mixture 200 based on a predefined G-code. By virtue of such implementations, on-demand gelation and patterning of prepolymer material 201 can be achieved while the unexposed regions of biopolymer mixture 200 stay uncrosslinked and can be removed. In some implementations, the transducer/radiation source can be moved along a trajectory. In other implementations, transducer arrays or acoustic holograms can be used to generate the pattern without the need for moving a transducer.

The imaging device 90 can employ any suitable imaging modality to visualize the biopolymer mixture before, during, and/or after bioprinting/gelation. Images generated by imaging device 90 could be used to guide the bioprinting device 100 when delivering radiation 115. In some implementations, imaging modalities that do not produce ionizing radiation, including ultrasound imaging or magnetic resonance imaging (MRI), can be employed. However, other suitable imaging modalities, including x-rays or computed tomography (CT) scans could potentially be employed with the techniques described herein.

To enable subcutaneous and deep tissue penetration of radiation 115 such that it reaches the target location for in vivo bioprinting, focused ultrasound waves can be used in some implementations. Among different radiations, ultrasound waves are well-known in medicine for low-risk diagnostics and therapeutics over many years. HIFU is a non-invasive treatment for cancer disease that uses high-frequency sound waves focused on the cancerous tissue to destroy the cancer cells by localized heating and ablation. As such, in some implementations, bioprinting device 100 can employ one or more HIFU transducers to deliver radiation 115 that causes carrier particles 202 to release encapsulated crosslinking agents 204. The HIFU transducers can be guided by imaging device 90 (e.g., using one or more ultrasound imaging transducers of imaging device 90) to enable controlled printing of functional biomaterials deep inside the body enabled by deep-penetrating HIFU waves. In such implementations, the biopolymer mixture 200 can be formulated as an “ultrasound ink” or “US-ink” with selected properties that are locally crosslinked through various chemical mechanisms upon focused ultrasound exposure. Depending on the application, the desired printing resolution, and/or the desired printing depth, the HIFU transducers can transmit HIFU waves using various suitable frequencies. For example, frequencies in the range of about 1 MHz to 10 MHz (e.g., 1.1 MHz, 2.65 MHz, and 8.75 MHz) were studied, but other frequencies could be used. In general, lower frequencies can lead to improved penetration. A resolution of about 150 μm was obtained for the transducer with 8.75 MHz frequency. However, it should be appreciated that the technology described herein is not limited to the frequencies and resolutions listed in this disclosure.

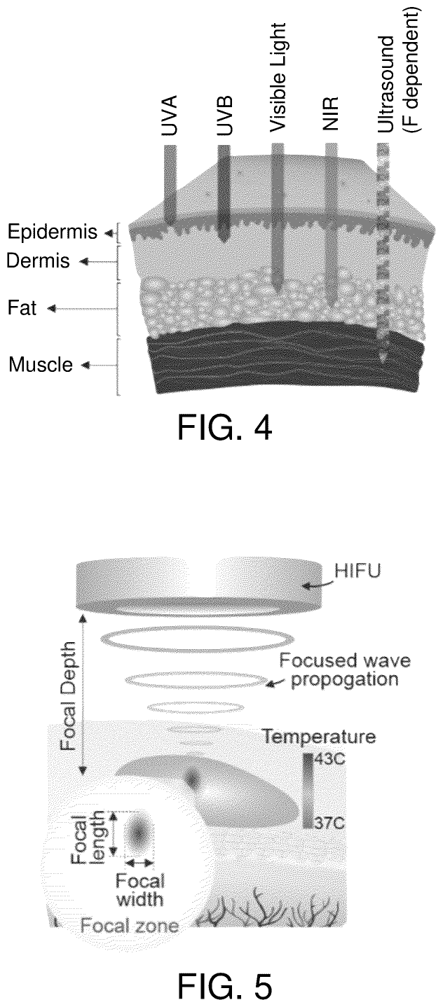

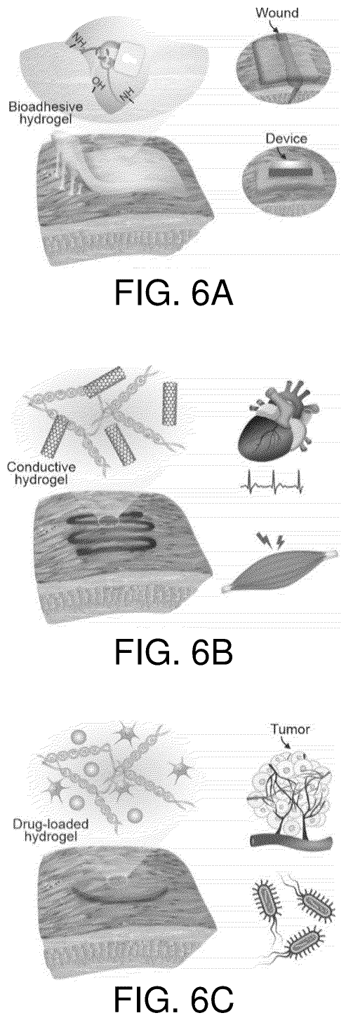

To appreciate the benefits of using HIFU energy in some implementations of the technology described herein, it is instructive to consider the potential penetration depth and printing resolution that can be achieved using focused ultrasound. For example, FIG. 4 schematically illustrates the tissue penetration depth that can be achieved by different types of light radiation as contrasted with ultrasound. Compared to light, ultrasound can penetrate much deeper (i.e., centimeters) into the tissue, including reaching muscle tissue. FIG. 5 schematically illustrates how the ultrasound focal point of HIFU can be used to define a high printing resolution. In addition to reaching deep tissue, HIFU can have focal points with sub-millimeter size diameters, enabling high-resolution in vivo printing. In some implementations, HIFU can be configured to heat the target location to a temperature between 39° C. and a 43° C. It should be noted that other sources of energy, other than HIFU energy, which can penetrate deep inside the body, could be used with the technology described herein.

In some implementations the prepolymer material 201 can comprise alginate, PEGDA, or PDMS monomers.

In some implementations, the contrast agent 203 can comprise gas vesicles (GVs).

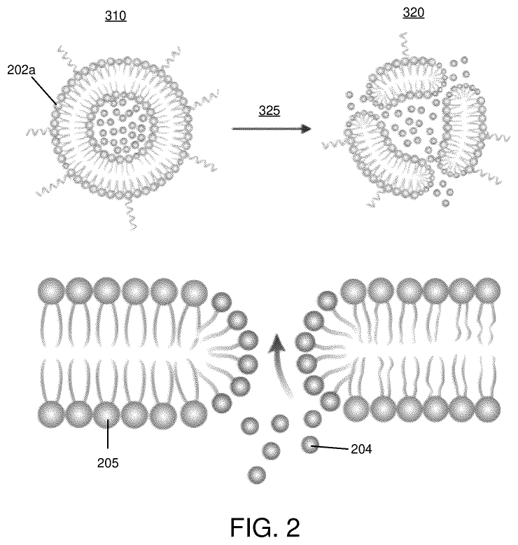

In some implementations, the carrier particles 202 are carrier vesicles encapsulating crosslinking agents 204. After radiation (e.g., ultrasound) exposure, the carrier vesicles can be defected and release the encapsulated crosslinking agent 204, triggering gelation of a matrix of prepolymer material 201. Liposomes, spherical vesicles composed of lipid bilayers, are one of the potential types of vesicles that can be triggered by radiation such as HIFU. Liposomes can be engineered to be thermosensitive and release the encapsulated material by a mild temperature increase induced by radiation such as HIFU. Exposing HIFU can induce hyperthermia in the focal point and enable releasing the crosslinking agent from the liposomes. This process is conceptually illustrated by FIG. 2, which is a schematic showing lipid bilayer changes of a liposome carrier 202a upon heating 325 from a solid state 310 to a liquid state 320. Upon HIFU or other radiation exposure, due to the mild temperature raise at the focal point, the lipid bilayer 205 of the liposome can change from the solid to liquid phase, leading to formation of nanopores on the lipid bilayer that release the crosslinking agent 204. In such implementations, the prepolymer material 201 can be formulated in a way to be liquid at body temperature of the subject (e.g., about 36-37.5° C. for humans) but have a gelation transition temperature of a few degrees above the body temperature.

It should be noted that the technology described herein can be applied to a wide range of materials (elastomers, hydrogels, ceramic or metallic composites, etc.). In addition, the technology described herein is not limited to liposomes as a delivery systems as other types of particles (e.g., bubbles, polymers, micelles, etc.) could also be used for delivering the crosslinking agent.

The crosslinking agents 204 can induce crosslinking of prepolymer material 201 using any suitable crosslinking mechanism. In some implementations, thermal crosslinking can be leveraged such that when the temperature locally increases at the focal point of radiation, this leads to the release of the crosslinking agent and in situ crosslinking. In addition to thermal crosslinking, various types of crosslinking mechanisms such as ionic crosslinking and free-radical polymerization can be employed. For example, calcium (Ca2+) ions can be encapsulated in liposomes to enable ionic gelation of alginate. In some implementations, materials that produce free radicals upon radiation exposure can be employed, eliminating the need for using carriers for the crosslinking agents 204.

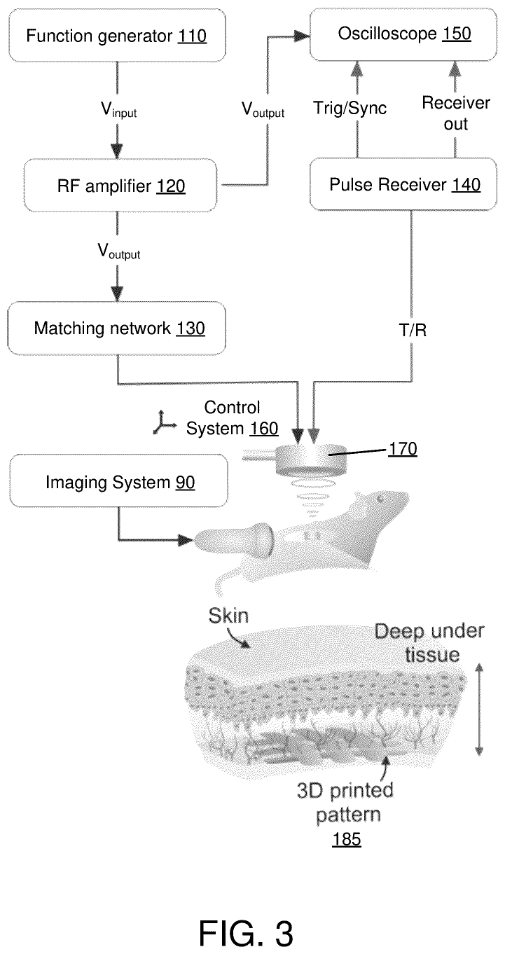

FIG. 3 illustrates example components that can be included with an in vivo bioprinting device 100 to enable deep tissue in vivo printing, in accordance with some implementations of the disclosure. As depicted, the bioprinting device 100 can include a function generator 110, an RF amplifier 120, a matching network 130, a pulse receiver 140, an oscilloscope 150, a control system 160, and one or more transducers 170. The function generator 110 can be configured to generate electrical signals to drive an ultrasound transducer 170, controlling the timing and frequency of acoustic pulses. The RF amplifier 120 can be configured to amplify the electrical signals from the function generator 110 to provide sufficient power to the ultrasound transducer 170 for effective bioprinting. The matching network 130 can be configured to optimize the impedance matching between the RF amplifier 120 and the ultrasound transducer 170 to maximize energy transfer and minimize reflections. The pulse receiver 140 can be configured to detect reflected ultrasound signals to find the focal point. The oscilloscope 150 can be configured to display the electrical signals generated, allowing visualization and analysis of the bioprinting process. Control system 160 can be configured to control the one or more transducers 170 that deliver energy to a deep tissue or subcutaneous target location to form a 3D printed gel pattern 185. In addition, an imaging device 90 can be used in conjunction with the in vivo bioprinting device 100 to monitor the prepolymer at the target location.

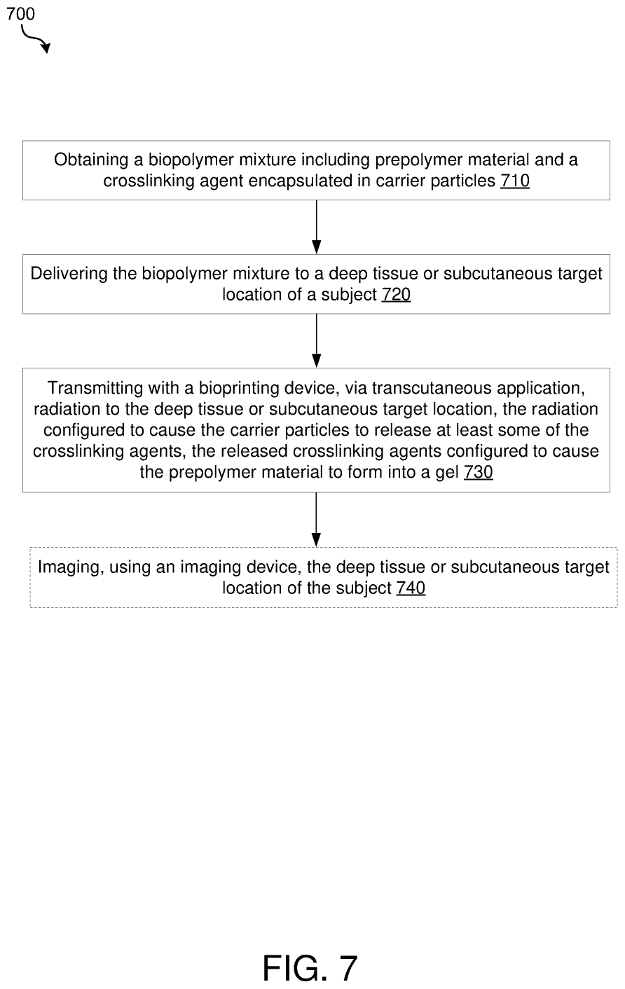

Prepolymer materials 201 having a wide variety of properties (e.g., conductive, bioadhesive, antibacterial, drug-loaded, etc.) could be incorporated into the biopolymer mixture 200 for use with the in vivo printing systems and methods described herein, enabling use of the technology in a wide variety of applications. For example, FIG. 6A is a schematic diagram illustrating an application of the in vivo printing technology described herein in wound sealing. In this implementation, the prepolymer material can be bioadhesive, and a hydrogel formed from the prepolymer material can be configured to seal an internal wound of the subject at the deep tissue or subcutaneous target location. FIG. 6B is a schematic diagram illustrating an application of the in vivo printing technology described herein in bioelectronics. In this implementation, the prepolymer material is electrically conductive, and a hydrogel formed from the prepolymer material is electrically conductive. FIG. 6C is a schematic diagram illustrating an application of the in vivo printing technology described herein in drug delivery and tissue regeneration. In this implementation, the prepolymer material can be loaded with drugs or growth factors. A hydrogel formed from the prepolymer material can be configured to provide a controlled release of drugs to tissue in the deep tissue or subcutaneous target location. Another potential application includes tissue engineering. For example, considering the mild temperature changes and biocompatibility of some implementations of the technology described herein, cell-encapsulated biomaterials could be crosslinked with this approach. When using smart inks as a prepolymer (e.g., thermoresponsive materials), radiation (e.g., HIFU) could be employed for both crosslinking the prepolymer followed by triggering the formed structure for further functions including shape deformation, movement, etc.

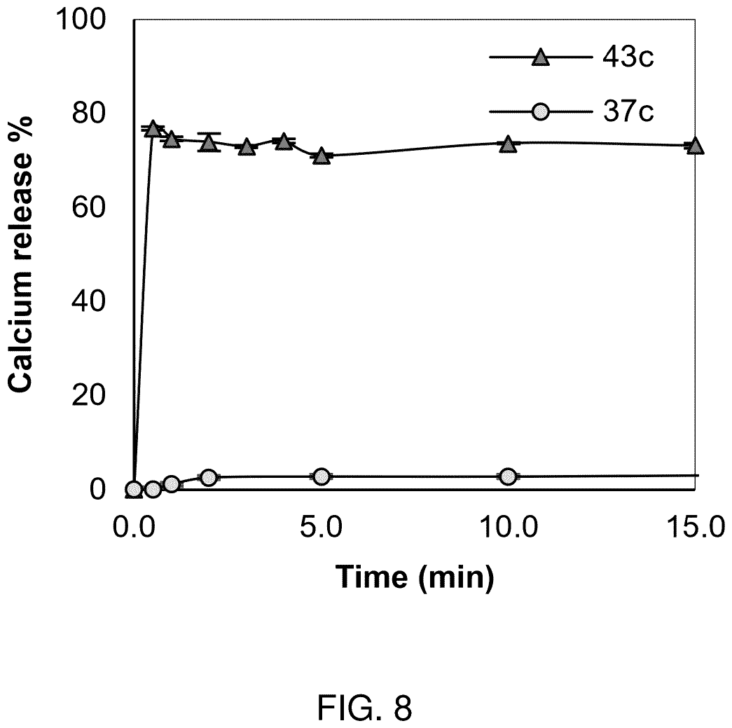

FIG. 7 is an operational flow diagram illustrating example operations performed by system 10 for deep tissue in vivo printing in a subject, in accordance with some implementations of the disclosure. Operation 710 includes obtaining a biopolymer mixture (e.g., biopolymer mixture 200) including prepolymer material and a crosslinking agent encapsulated in carrier particles. In some implementations, the biopolymer mixture can also contain a contrast agent to enhance imaging of a target location by an imaging device.

In some implementations, the crosslinking can be induced upon energy exposure without the need for carriers to deliver the crosslinking agent.

Operation 720 includes, delivering the biopolymer mixture to a deep tissue or subcutaneous target location of the subject. For example, the biopolymer mixture could be injected to a deep tissue target site such as an internal organ, muscle tissue, nervous tissue, or some other tissue site.

Operation 730 includes transmitting with an in vivo bioprinting device 100, via transcutaneous application, radiation to the deep tissue or subcutaneous target location, the radiation configured to cause the carrier particles to release at least some of the crosslinking agents, the released crosslinking agents configured to cause the prepolymer material to form into a gel. The radiation can be transmitted with one or more transducers of the bioprinting device, along a predetermined trajectory, to cause the prepolymer material to form into a pattern of the gel defined by the predetermined trajectory. In some implementations, the bioprinting device includes an array of transducers, and a controller of the bioprinting device is configured to control the array to operate the transducers in an order associated with the predetermined trajectory. In some implementations, the bioprinting device includes a motor configured to position the one or more transducers in relation to the deep tissue or subcutaneous target location, and a controller of the bioprinting device is configured to control the motor to position the one or more transducers to transmit, along the predetermined trajectory, the radiation to the deep tissue or subcutaneous target location.

Optional operation 740 includes imaging, using an imaging device, the deep tissue or subcutaneous target location of the subject. Operation 740 could be performed prior to, concurrently with, and/or after operation 730.

### Experimental Results

Various bioprinting experiments and studies were performed using a HIFU-based printing system and liposomes, in accordance with particular embodiments of the disclosure. Although these experimental results exemplify some of the advantages of utilizing the technology described herein, it should be appreciated that the disclosure is not limited by the discussion that follows, which describes results and observations of utilizing particular example embodiments. For example, the deep tissue in vivo bioprinting technology described herein need not necessarily be implemented using HIFU radiation and/or carrier liposomes.

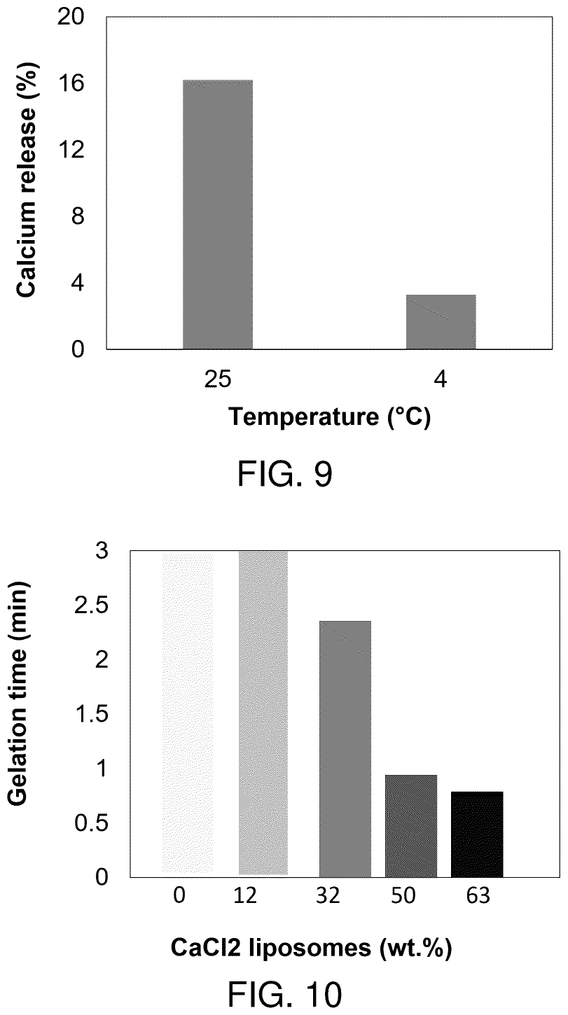

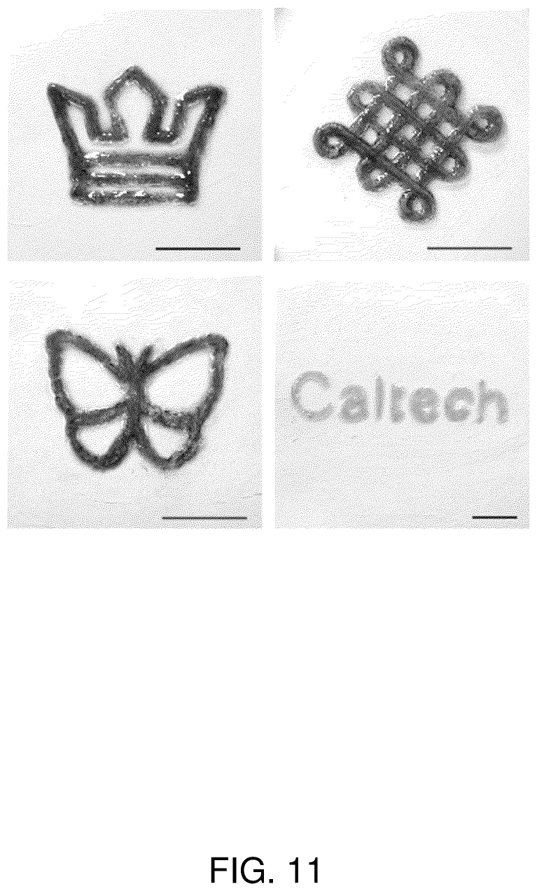

Liposomes were synthesized using the Bangham method, involving the formation of a dry lipid film, rehydration in a solution of crosslinking agent, agitation, and subsequently undergoing 10 cycles of extrusion to size the liposomes. FIG. 8 includes two plots showing calcium release over time upon heating liposomal solutions at 37° C. and 43° C. The liposomes showed approximately 80% release at 43° C. (bilayer in liquid phase) while no significant release at 37° C. (bilayer in solid phase), around body temperature, implicating the on-demand release as demonstrated in FIG. 8. The insignificant release of the liposomes (less than 3%) over the course of study enabled the successful application of an US-ink for in vivo printing. In addition, the long-term stability of the liposomes was studied over six months as shown in FIG. 9, which is a plot showing the calcium release from liposomes after 6 months of storage at 25° C. and 4° C. At room temperature, the liposomes had a calcium release of approximately 16%, while at 4° C. the liposomes had a calcium release of approximately 3%.

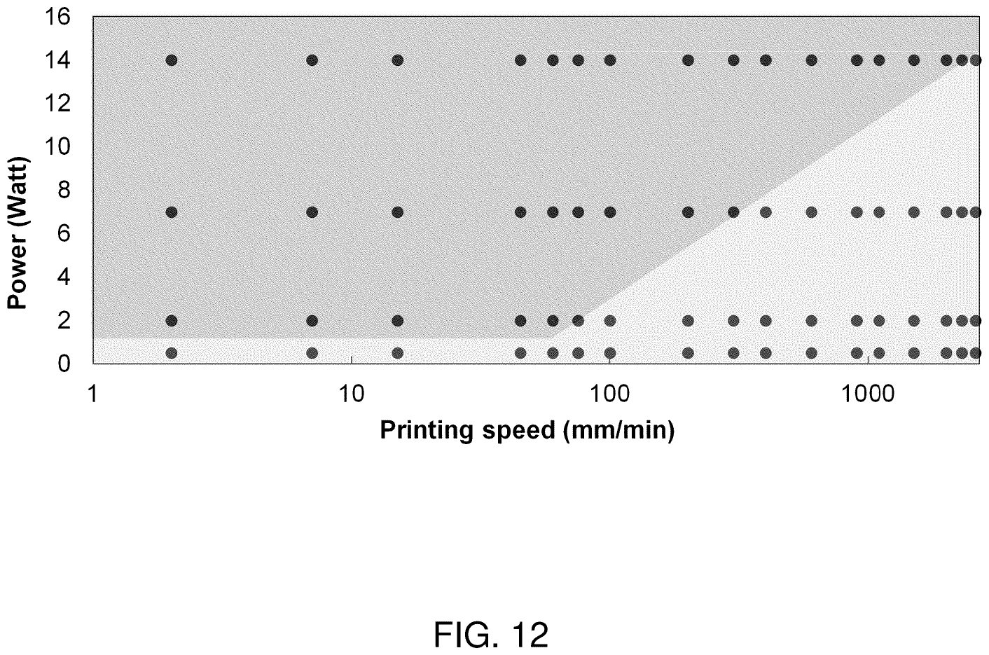

FIG. 10 includes a plot corresponding to a rheological study showing heat-trigged gelation of an alginate prepolymer for various liposome concentrations. In this study, the mixture was heated using a heating plate. The rheological study showed a liposome concentration of about 50 wt. % enabled gelation in less than 1 minute, and at the same time no significant release at body temperature. It should be noted that the final gelation time for all the liposome concentrations exposed to HIFU is shorter than what was obtained through the rheological study. This can be attributed to the direct heating at focal point in the HIFU. By contrast, the rheological study relied on heat transfer from one side (bottom plate). A similar study for a PEGDA-based biopolymer mixture confirmed the possibility of using this technology not only for biopolymers crosslinked through ionic crosslinking but also for free radical polymerization.

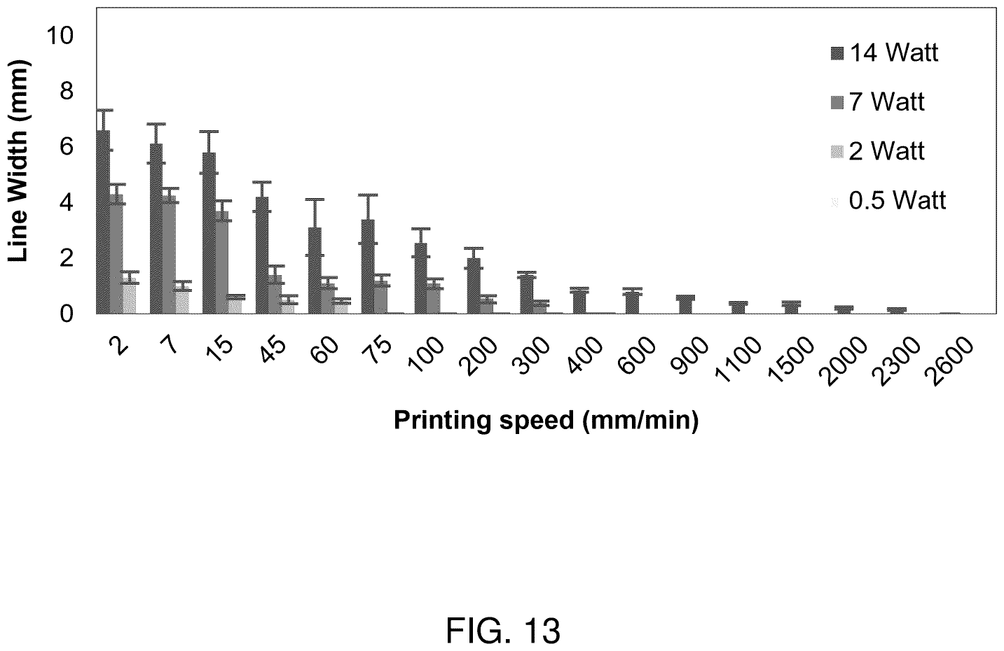

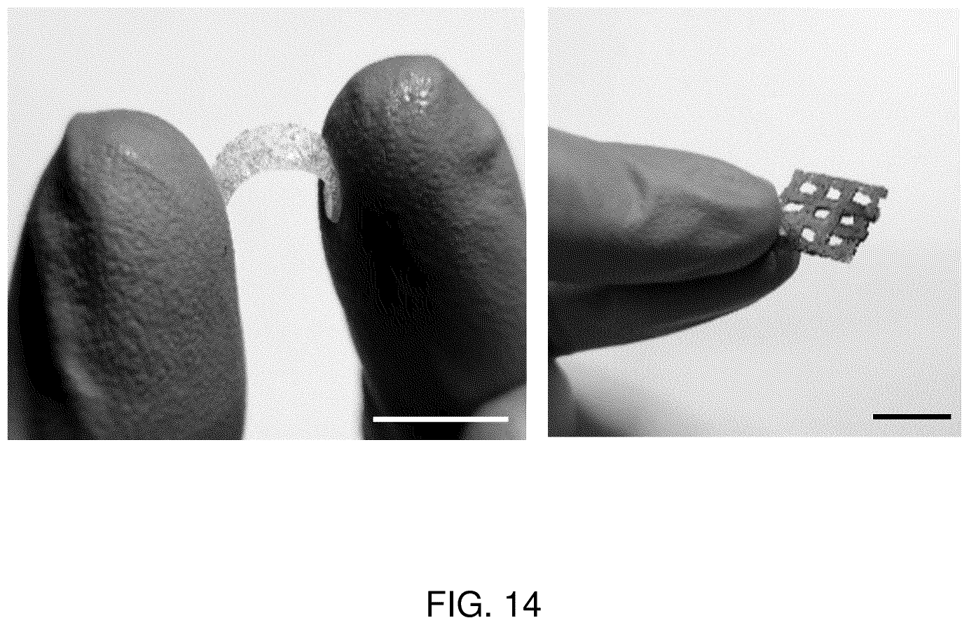

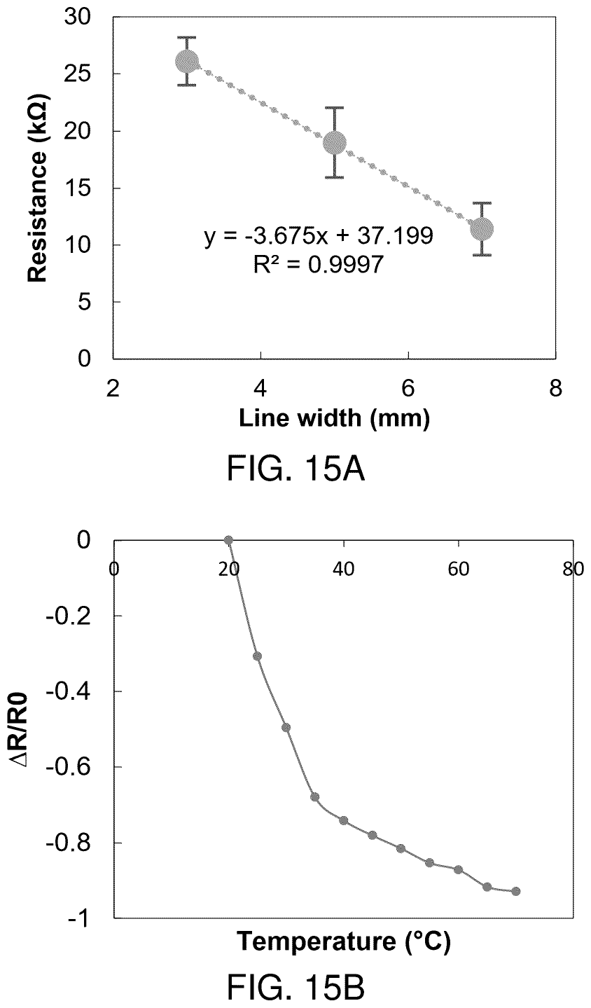

FIG. 11 shows images of various PDMS patterns 3D printed using HIFU. The scales are 1 cm. It was observed that different combinations of power and transducer movement speed in the in vivo bioprinting device could either result in the formation or absence of gel patterns. This is illustrated by FIG. 12, which is a plot showing power (Watt) and printing speed (mm/min) conditions leading to gel formation in the case of alginate prepolymer mixed with 50 wt. % CaCl2-encapsulated liposomes, where dark gray represents gel formation, and light gray represents no gel. It was also observed that power and printing speed conditions could be used to control or define the width (meaning the axial resolution) of the printed lines of the gel. This is illustrated by FIG. 13, which is a plot showing 3D bioprinting resolutions obtained in the case of alginate prepolymer mixed with 50 wt. % CaCl2-encapsulated liposomes using various power and printing speeds. In this experiment, the highest resolution was achieved at a printing speed of 2300 mm/min using a power of 14 watts.

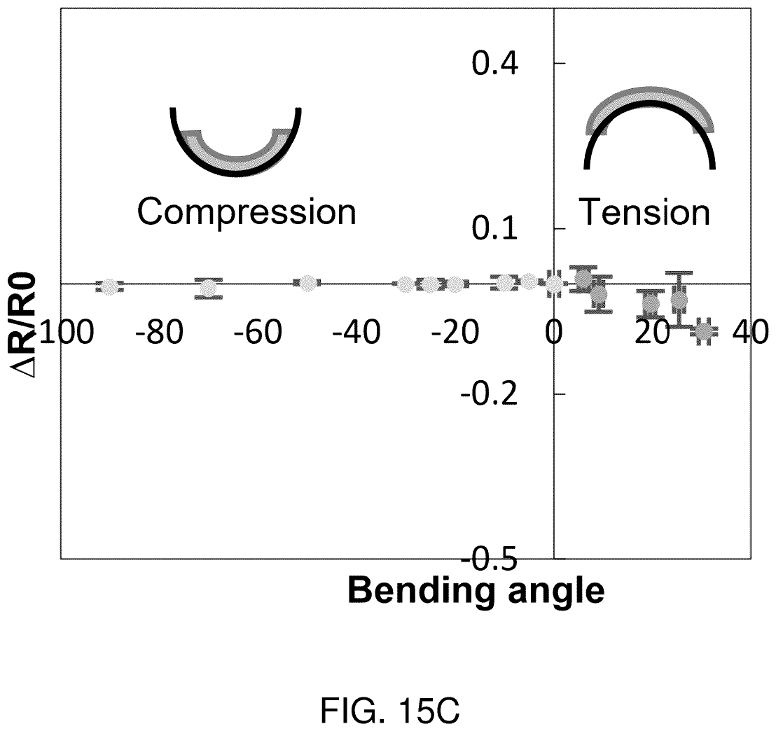

Other than alginate, other types of hydrogel prepolymers were prepared for 3D printing using this technology. For instance, FIG. 14 illustrates PEGDA 3D printed by ultrasound exposure. The scale bars are 1 cm. In this experiment, prepolymers composed of PEGDA monomer with 0.5 wt. % APS and 0.005 wt. % TEMED (encapsulated in liposomes) were prepared, and 3D printed. Encapsulating TEMED in liposomes can prevent long-term crosslinking over time.

It was also experimentally observed that ultrasound can lead to microjetting of the prepolymer into the underneath tissue and in situ crosslinking. This can eventually trigger a mechanical interlock of the hydrogel within the tissue and ultrasound-induced adhesion. A wide range of functional biomaterials (i.e., bioadhesives, bioelectronics, biocarriers, etc.) were introduced that could be crosslinked and patterned by the technology described herein.

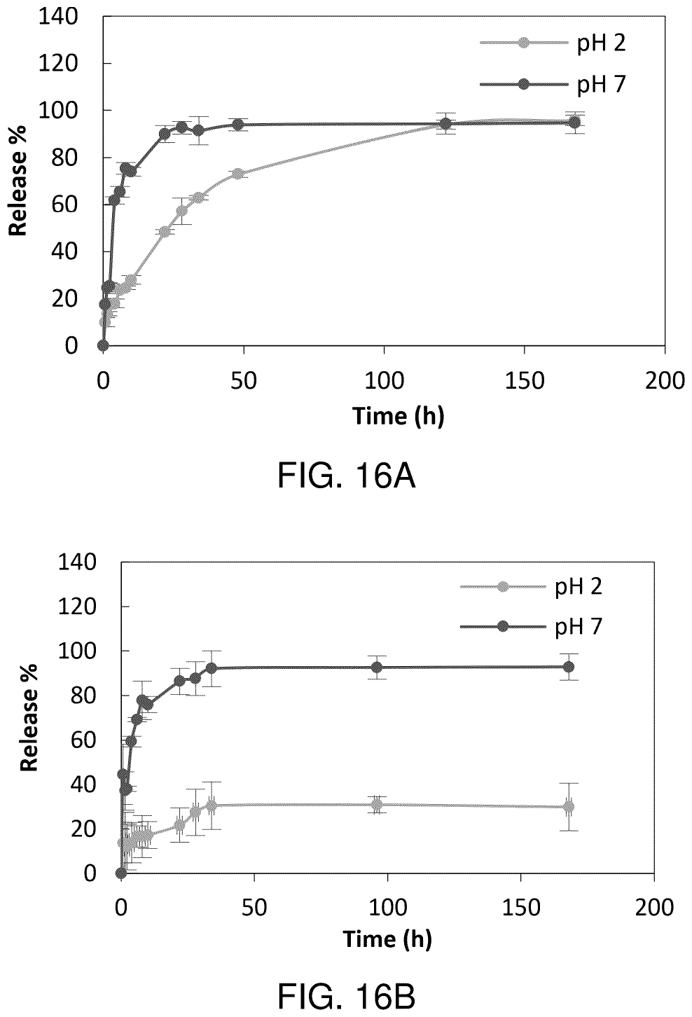

It was experimentally observed that a conductive biomaterial produced using the technology described herein could be used for sensing applications. For example, FIGS. 15A-15C are plots showing electromechanical properties of a conductive polymer 3D printed using HIFU. FIG. 15A includes a plot showing a resistance change for various line widths. As depicted, the developed conductive material showed a decrease in resistance as the line width increased. FIG. 15B includes a plot showing the ratio of resistance changes upon heating to different temperatures, illustrating the temperature sensitivity of the ultrasound-induced 3D printed conductive hydrogel. FIG. 15C includes plots showing 3D printed sensor stability under compression and tension. As depicted, the electrical properties of the 3D printed sensors were stable under compressive and tensile loadings scenarios induced by bending.

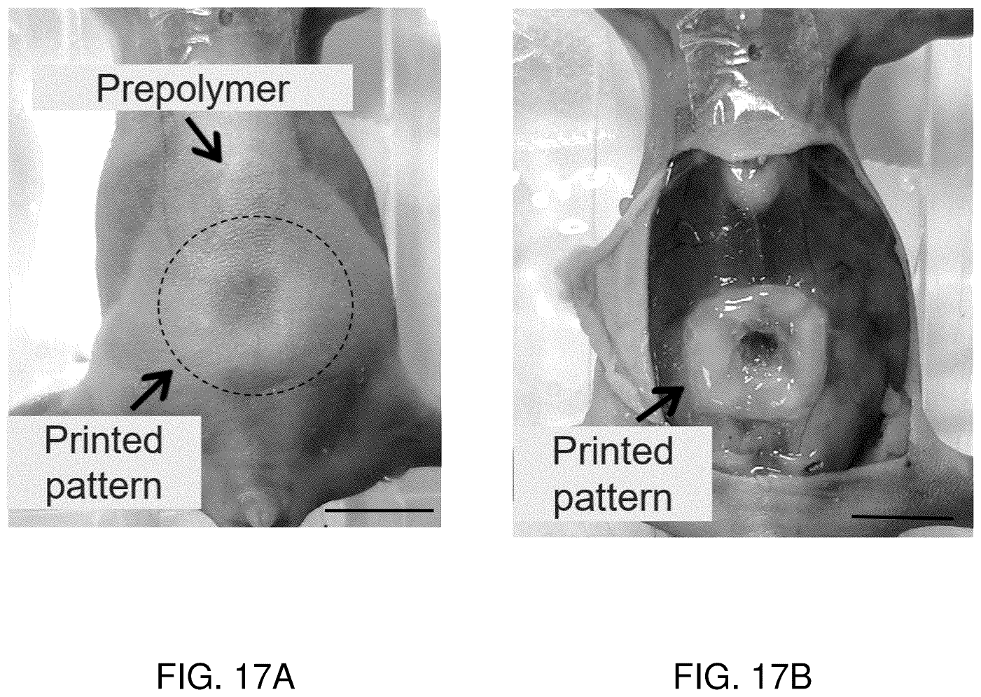

It was also experimentally observed that a prepolymer could be used as a biocarrier for a wide range of small and large molecules, including for 3D printing of drug-loaded polymers to have a sustained release over time. For example, FIGS. 16A-16B show small (FIG. 16A) and large (FIG. 16B) molecule release percentage as a function of time from HIFU induced 3D printed drug-loaded hydrogels under acidic and neutral pH. These examples illustrate drug loading and sustained release of the Rhodamine B and bovine serum albumin (BSA) from the crosslinked hydrogels. The examples also illustrate that the small and large molecules could have different release rates at acidic and neutral pH.

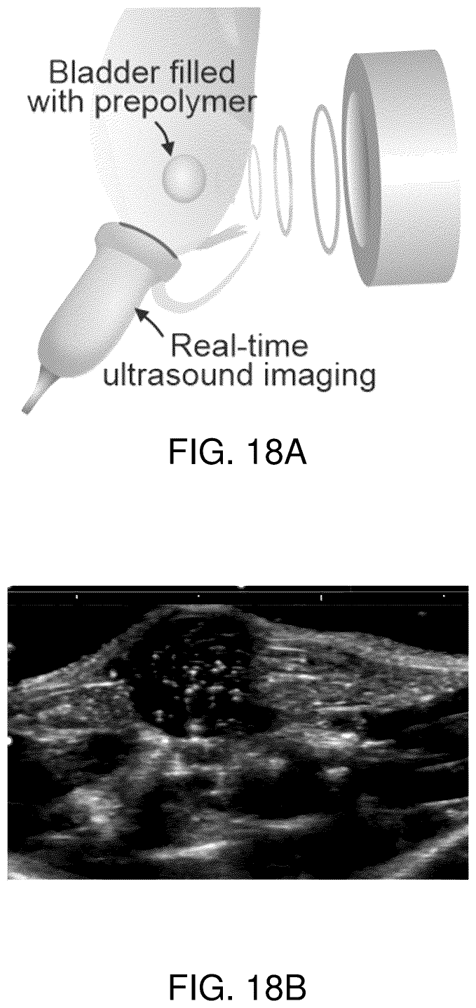

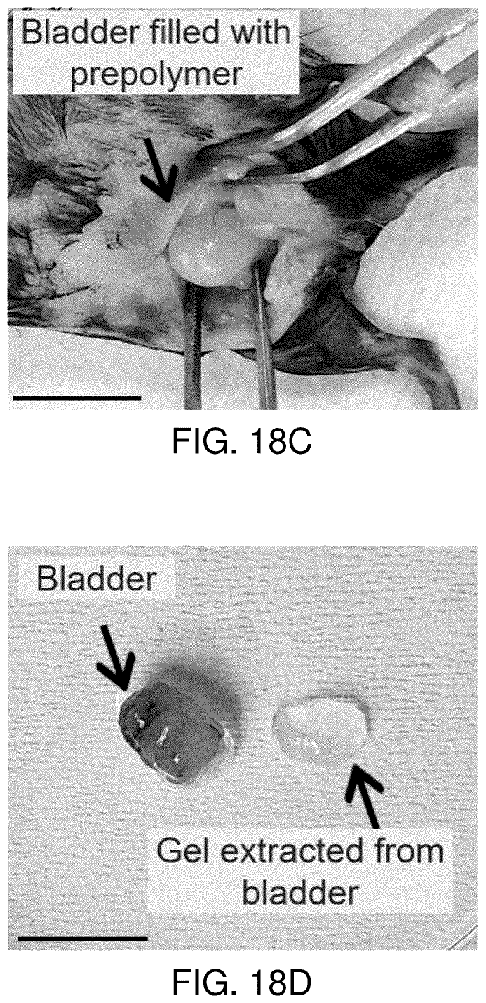

Experiments were conducted to 3D printed polymers deep inside the body confirmed by in vivo mouse models. FIGS. 17A-17B illustrate in vivo 3D printing of biomaterial deep inside the body of a mouse model using the technology described herein. FIG. 17A illustrates the injection site of a biopolymer mixture including a prepolymer under the mouse's skin. FIG. 17B illustrates the results of ultrasound exposure and 3D printing of a pattern. In this example, scale bars are 1 cm. The prepolymer was first injected under the skin followed by externally applying focused ultrasound waves to crosslink the material and make patterns. This technology was further demonstrated by ultrasound imaging-guided in vivo 3D printing inside a bladder, as illustrated by FIGS. 18A-18D. FIG. 18A is a schematic diagram showing in vivo printing inside the bladder. A prepolymer was applied into the bladder through instillation by a catheter (FIG. 18C). The process was minimally invasive, and incision was conducted only for the sake of taking a picture as a confirmation to show prepolymer had been successfully applied into the bladder. A contrast agent was mixed into the prepolymer to ensure visibility of the prepolymer inside the body using ultrasound imaging (FIG. 18B). The HIFU transducer was in the correct position regarding the bladder and imaging system and focused ultrasound waves were applied to the prepolymer. FIG. 18D shows the extracted bladder and gel. After 3D printing and crosslinking of the hydrogel, the bladder was extracted to show successful printing and gelation. Scale bars are 1 cm.

As the foregoing experiments illustrate, the technology could be applied to a wide range of in vivo applications, including treatment of cancers such as acute bladder cancer. For example, drug-loaded prepolymers could be 3D printed/gelated in a tumor site for sustained release of anti-cancer drugs.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A method, comprising:
obtaining a biopolymer mixture including prepolymer material and a crosslinking agent encapsulated in carrier particles;
delivering the biopolymer mixture to a subcutaneous or deep tissue target location of a subject; and
transmitting with a bioprinting device, via transcutaneous application, radiation to the subcutaneous or deep tissue target location, the radiation configured to cause the carrier particles to release at least some of the crosslinking agent, the released crosslinking agent configured to cause the prepolymer material to form into a gel or polymeric matrix.

2. The method of claim 1, wherein:
the biopolymer mixture further includes a contrast agent; and
the method further comprises: capturing, using an imaging device, an image of the subcutaneous or deep tissue target location, the image enhanced by the contrast agent.

3. The method of claim 1, wherein the carrier particles comprise vesicles, micelles, bubbles, or polymers.

4. The method of claim 3, wherein:
the vesicles comprise liposomes encapsulating the crosslinking agent; and
the radiation is configured to cause the liposomes to increase in temperature and release the crosslinking agent.

5. The method of claim 1, wherein:
the bioprinting device comprises one or more high-intensity focused ultrasound (HIFU) transducers; and
transmitting with the bioprinting device, via transcutaneous application, the radiation to the subcutaneous or deep tissue target location comprises: transmitting with the one or more HIFU transducers, HIFU to the subcutaneous or deep tissue target location, the HIFU configured to cause the carrier particles to increase in temperature and release at least some of the crosslinking agent.

6. The method of claim 5, wherein the HIFU is configured to heat the subcutaneous or deep tissue target location to between 39° C. and 43° C.

7. The method of claim 1, wherein:
the prepolymer material is loaded with drugs or cells; and
the gel formed from the prepolymer material is configured, in the subcutaneous or deep tissue target location, to provide a controlled release of the drugs for a therapeutic application or to encapsulate the cells for tissue regeneration.

8. The method of claim 1, wherein:
the prepolymer material is electrically conductive; and
the gel formed from the prepolymer material is electrically conductive.

9. The method of claim 1, wherein:
the prepolymer material is bioadhesive; and
the gel formed from the prepolymer material is configured to seal an internal wound of the subject at the subcutaneous or deep tissue target location.

10. The method of claim 1, wherein transmitting with the bioprinting device, via transcutaneous application, the radiation to the subcutaneous or deep tissue target location comprises: transmitting with one or more transducers of the bioprinting device, along a predetermined trajectory, the radiation to the subcutaneous or deep tissue target location to cause the prepolymer material to form into a pattern of the gel defined by the predetermined trajectory.

11. A system for in vivo bioprinting, the system comprising:
a biopolymer mixture including prepolymer material and a crosslinking agent encapsulated in carrier particles; and
a bioprinting device configured to transmit, via transcutaneous application, radiation to a subcutaneous or deep tissue target location of a subject that the biopolymer mixture is delivered to, the radiation configured to cause the carrier particles to release at least some of the crosslinking agent, the released crosslinking agent configured to cause the prepolymer material to form into a gel or polymeric matrix.

12. The system of claim 11, wherein:
the biopolymer mixture further includes a contrast agent; and
the system further comprises an imaging device configured to capture an image of the subcutaneous or deep tissue target location of the subject after the biopolymer mixture is delivered to the subcutaneous or deep tissue target location.

13. The system of claim 12, wherein:
the bioprinting device comprises one or more high-intensity focused ultrasound (HIFU) transducers, the one or more HIFU transducers configured to transmit the radiation as HIFU to the subcutaneous or deep tissue target location, the HIFU configured to cause the carrier particles to release at least some of the crosslinking agent; and
the imaging device comprises an ultrasound imaging device.

14. The system of claim 11, wherein the bioprinting device comprises:
one or more transducers configured to the transmit the radiation to the subcutaneous or deep tissue target location; and
a controller configured to cause the one or transducers to transmit, along a predetermined trajectory, the radiation to the subcutaneous or deep tissue target location to cause the prepolymer material to form into a pattern of the gel defined by the predetermined trajectory.

15. The system of claim 14, wherein:
the bioprinting device comprises an array of transducers including the one or more transducers; and
the controller is configured to cause the transducers of the array of transducers to operate in an order associated with the predetermined trajectory.

16. The system of claim 14, wherein:
the bioprinting device comprises a motor configured to position the one or more transducers in relation to the subcutaneous or deep tissue target location; and
the controller is configured to control the motor to position the one or more transducers to transmit, along the predetermined trajectory, the radiation to the subcutaneous or deep tissue target location.

17. The system of claim 11,
the carrier particles comprise temperature sensitive particles configured to release the crosslinking agent in response to a temperature increase; and
the crosslinking agent is configured to induce thermal crosslinking of the prepolymer material.

18. The system of claim 11, wherein:
the prepolymer material is loaded with drugs, and the gel formed from the prepolymer material is configured to provide a controlled release of the drugs in the subcutaneous or deep tissue target location; or
the prepolymer material is loaded with cells, and the gel formed from the prepolymer material is configured to encapsulate the cells for tissue regeneration in the subcutaneous or deep tissue target location; or
the prepolymer material is electrically conductive, and the gel formed from the prepolymer material is electrically conductive; or
the prepolymer material is bioadhesive, and the gel formed from the prepolymer material is configured to seal an internal wound of the subject at the subcutaneous or deep tissue target location.

19. A biopolymer mixture for in vivo bioprinting, the biopolymer mixture comprising:
a contrast agent configured to enhance an image of the biopolymer mixture;
a crosslinking agent encapsulated in carrier particles, the carrier particles configured to release the crosslinking agent in response to a temperature increase; and
a prepolymer material configured to form into a gel when exposed to the crosslinking agent.

20. The biopolymer mixture of claim 19, wherein the carrier particles comprise vesicles, micelles, bubbles, or polymers.
